SEQUENCE LISTING (4 sequences)

SEQ ID NO: 1 - protein, 122 aa, Homo sapiens
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSS

SEQ ID NO: 2 - protein, 107 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIK

SEQ ID NO: 3 - protein, 452 aa, Homo sapiens
QVQLVQSGAEVKKPGSSVKVSCKASGGTFSSLAISWVRQAPGQGLEWMGGIIPIFGTANYAQKFQGRVTITADESTSTAYMELSSLRSEDTAVYYCARGGSVSGTLVDFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 214 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNIYPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0094212 A1
(43) Pub. Date: Mar. 21, 2024

(54) NOVEL BIOMARKERS AND USES THEREOF

(71) Applicant: Hoffmann-La Roche Inc., Little Falls, NJ (US)

(72) Inventors: Michael CANNARILE, München (DE); Bruno GOMES, Flaxlanden (FR); Vaios KARANIKAS, Adliswil (CH); Theresa KOLBEN, Muenchen (DE); Dominik RUETTINGER, Seehausen (DE); Fabian SCHMICH, Bad Feilnbach (DE)

(73) Assignee: Hoffmann-La Roche Inc., Little Falls, NJ (US)

(21) Appl. No.: 18/459,135

(22) Filed: Aug. 31, 2023

Related U.S. Application Data
(63) Continuation of application No. PCT/EP2022/054930, filed on Feb. 28, 2022.

(30) Foreign Application Priority Data
Mar. 1, 2021 (EP) 21160004.4

(51) Int. Cl. G01N 33/574; C07K 16/28; C12Q 1/6886

(52) U.S. Cl. CPC G01N 33/57484; C07K 16/2866; C12Q 1/6886; C12Q 2600/106; C12Q 2600/158

(57) ABSTRACT

Disclosed herein are novel biomarkers for identifying patients, with cancer, likely to benefit from treatment with an anti-CD25 agent. Also disclosed are methods using said biomarkers for making a treatment decision—or monitoring treatment with an anti-CD25 agent as well as methods of treating a patient, with cancer, comprising administering an anti-CD25 agent based on prior use of the present biomarkers.

### CROSS REFERENCE TO RELATED APPLICATIONS

This application is a continuation of International Application No. PCT/EP2022/054930, filed Feb. 28, 2022, which claims priority to European Patent Application No. 21160004.4 filed Mar. 1, 2021, each of which is incorporated herein by reference in its entirety.

### SEQUENCE LISTING

This application contains a Sequence Listing which has been submitted electronically in XML format and is hereby incorporated by reference in its entirety. Said XML copy, created on Oct. 10, 2023, is named P36746-US Sequence Listing.xml and is 5.18 KB bytes in size.

### FIELD OF INVENTION

The present invention is generally related to the field of biomarkers for use to enable treatment decision or patient stratification, and in particular for enabling treatment decision for patients in the field of oncology.

Since their discovery, Tregs have been found to be critical in mediating immune homeostasis and promoting the establishment and maintenance of peripheral tolerance. However, in the context of cancer their role is more complex. As cancer cells express both self- and tumour-associated antigens, the presence of Tregs, which can dampen effector cell responses, can contribute to tumour progression. The infiltration of Tregs in established tumours therefore is considered as one of the main obstacles to effective anti-tumour responses and to treatment of cancers in general. Suppression mechanisms employed by Tregs are thought to contribute significantly to the limitation or even failure of current therapies, in particular immunotherapies that rely on induction or potentiation of anti-tumour responses (Onishi H et al; 2012).

CD25 is one of the potential molecular targets for achieving depletion of Tregs. Indeed, CD25, also known as the interleukin-2 high-affinity receptor alpha chain (IL-2Rα), is constitutively expressed at high-levels by Treg cells, and low-levels by T effector cells and is thus a promising target for Treg depletion. Antibodies targeting CD25, especially non IL2 blocking anti-CD25 antibodies are known in the art, and are for example disclosed in WO2018/167104 or WO2019/175222.

In humans, high tumor infiltration by Treg cells and, more importantly, a low ratio of effector T (Teff) cells to Treg cells, is associated with poor outcomes in multiple cancers. Conversely, a high Teff/Treg cell ratio is associated with favourable responses to immunotherapy (M. Amann, S. A. Quezada et al, Nature Cancer, Vol. 1, 2020, 1153-1166.). Nevertheless, depletion of Tregs in tumours is complex, and results of preclinical and clinical studies in this area had been inconsistent, mostly due to the difficulty of identifying a target specific for Treg.

Moreover, one of the challenges of identifying a patient enrichment strategy for the use of Treg depleters (more specifically, anti-CD25 antibodies), or a patient population likely to benefit from treatment with those agents, is that the exact mode of action regarding anti-tumor immunity for such a new modality is still unknown. There remains thus a need to identify biomarkers useful for identifying patients which are likely to benefit from treatment with anti-CD25 antibodies. Such biomarkers and patient enrichment strategies may also help avoiding unnecessary treatment burden and to thus enable treatment dictions.

### SUMMARY OF THE INVENTION

The present invention provides a biomarker for identifying patients likely to benefit from treatment with an anti-CD25 agent, wherein such biomarker consists in identifying the immune phenotype of a patient's tumor as being the inflamed immune phenotype, preferably CD8+ inflamed immune phenotype.

The present invention also provides a method of identifying a patient having cancer as likely to respond to a therapy comprising an anti-CD25 agent, the method comprising the use of the biomarker according to the present invention prior to starting treatment with said anti-CD25 agent (at baseline). This method may be an in vitro method.

The present invention also provides a method of treating a patient suffering from cancer with an anti-CD25 agent, said method comprises using the biomarker in accordance with the present invention to assist making that treatment decision.

The present invention also provides an anti-CD25 agent for use in treating a patient having cancer, wherein the patient is selected for treatment based on detection of the biomarker in accordance with the present invention in a sample from the patient at baseline (i.e. prior to treatment with said anti-CD25 agent).

The present invention also provides a method for monitoring treatment of a patient with an anti-CD25 agent, wherein recommendation to continue the treatment is based on detecting the biomarker according to the present invention in a sample from that patient during treatment.

In accordance with the present invention, the patient is a human suffering from cancer.

### BRIEF DESCRIPTION OF THE FIGURES

FIG. 1: The concept of Patient enrichment strategies in early clinical development. Patient enrichment aims at optimising decision making in Phase 1 clinical experiments. The aim is to identify high confidence markers in responding or non-responding patients to increase the Effect vs no Effect ratio compared to the all corner approach using also surrogate enrichment markers. Such approaches may combine, high confidence and lower confidence enrichment elements

FIG. 2: FOXP3 content determined by IHC across indications. Internal databases were used to visualise presence of FOXP3 cells by IHC. FOXP3 cell numbers (median) across indications showed high variability with the highest observed in HNSCC, Melanoma and NSCLC.

FIG. 3: Internal database analyses support patient selection by immune phenotype as surrogate for FOXP3. Samples from Roche internal tissue/sample databases were interrogated with respect to relation of FOXP3 and CD8 expression against line of treatment (FIG. 3a), Immune phenotype (FIG. 3b), PDL1 expression status (FIG. 3c), and TMB status (FIG. 3d)

FIG. 4: Internal database analyses support patient selection by immune phenotype as surrogate for FOXP3.

### SEQUENCE LISTINGS

SEQ ID NO:1 represents the heavy chain variable domain (VH) amino acid sequence of an anti-CD25 antibody in accordance with the present invention.

SEQ ID NO:2 represents the light chain variable domain (VL) amino acid sequence of an anti-CD25 antibody in accordance with the present invention.

SEQ ID NO:3 represents the heavy chain (HC) amino acid sequence of an anti-CD25 antibody in accordance with the present invention.

SEQ ID NO:4 represents the light chain (LC) amino acid sequence of an anti-CD25 antibody in accordance with the present invention.

### DETAILED DESCRIPTION OF THE INVENTION

The vast majority of Phase 1 oncology trials use an all-corner population approach. One of the advantages of such an approach is the fast recruitment. However, the number of patients recruited per indication is low due to the overall low sample size. Therefore, large decisions (budget, molecule go/no-go) are based on small and heterogeneous patient sample sets, which bear a high risk of false negative decision-making. To increase the quality of early clinical development decisions by increasing sensitivity and specificity of decisions, the present inventors have implemented a stringent approach to patient enrichment for cancer patients undergoing treatment with an anti-CD25 antibody. The approach of incorporating the conceptual integration of direct and surrogate selection markers, was undertaken in order to:
i. increase the success of Proof of Concept (PoC) studies,
ii. enable definitive experiments,
iii. improve quality of decision-making and decrease the time to decision, and
iv. serve as an early lead for a biomarker and companion diagnostic strategy by identifying the patient population most likely to benefit.

CD8 T cell levels within the tumor microenvironment (TME) have long been implicated as a surrogate biomarker of response to immunotherapy (1). Although it is unknown why some tumors have a higher CD8 infiltration than others, several reasons have been identified as probable drivers of this, such as tumor mutational burden, stromal and myeloid cell presence, inflammatory signals and others (2).

More recently, the location of such CD8 T cells has been shown to define ever further the inflammatory status of any given tumor into inflamed and non-inflamed (3, 4). Inflamed tumors appear to be predominated by high densities of tumor-infiltrating lymphocytes (TIL) and IFNγ-producing CD8+ T cells, expression of PD-L1 in tumor-infiltrating immune cells, and the presence of a preexisting antitumor immune response. In contrast, non-inflamed tumors are immunologically ignorant and are poorly infiltrated by lymphocytes, rarely express PD-L1, and are characterized by highly proliferating tumors with low mutational burden and low expression of antigen presentation machinery markers (5, 6). Thus, such tumor characteristics can be grouped into three immune profiles, depending on their immune status—called immune phenotypes. These immune profiles have been termed immune desert-, immune-excluded—and inflamed tumors (7) and have been used to monitor their relation to CIT treatment (8).

The present inventors hypothesized that, in order to show anti-tumor effects following Treg depletion due to administration of an anti-CD25 antibody, patients with the highest FOXP3 expression levels as a surrogate marker for Tregs needed to be identified. Since no formal cut-off for FOXP3 expression exists to date, alternative markers associated with high FOXP3 expression were investigated. The influence of tumor type, prior anti-cancer treatments, PD-L1 status, tumor mutational burden (TMB) and immune phenotype as they related to FOXP3 expression were analysed in a joint linear model. Interestingly, the CD8 inflamed immunophenotype (intratumoral CD8 infiltration) stood out as the factor most strongly associated with high FOXP3 expression (p<0.01). The present inventors also expand enriching for patients with an inflamed phenotype as a surrogate for high FOXP3/Treg prevalence to a study combining an anti-CD25 antibody with atezolizumab. Moreover, monitoring treatment-related immune phenotype changes can inform on further treatment decisions.

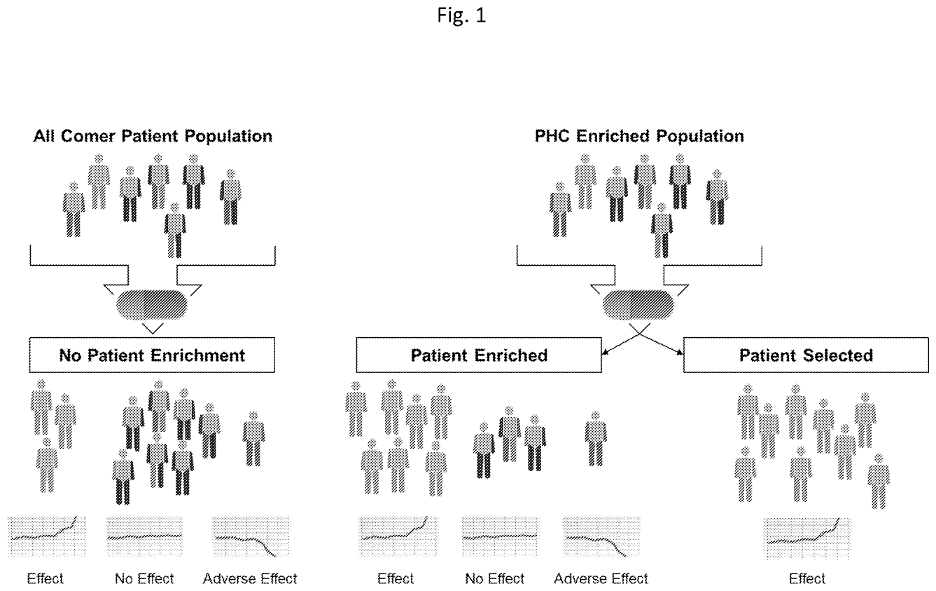

Patient enrichment strategies are a means to reduce the risk of false negative or positive decision making and eventually lead to early trial attrition. Patient enrichment aims at optimising decision making in Phase 1 clinical experiments. The aim is to identify high confidence markers in responding or non-responding patients to increase the Effect vs no Effect ratio compared to the all corner approach using surrogate enrichment markers (FIG. 1). Such approaches may combine high confidence and lower confidence enrichment elements. Such considerations can lead to fit for purpose strategies tailored to specific drugs.

Therefore, in one embodiment, the present invention provides a biomarker for identifying patients likely to benefit from (or respond to) treatment with an anti-CD25 agent, wherein such biomarker consists in identifying the immune phenotype of a patient's tumor as being the inflamed immune phenotype, preferably CD8+ inflamed immune phenotype.

In another embodiment, the present invention provides a method of identifying a patient having cancer as likely to respond to a therapy comprising an anti-CD25 agent, the method comprising:
a) detecting the tumor immune phenotype in a sample from the patient;
b) comparing the result measured in a) to a reference level;
c) identifying the patient as likely to respond to the therapy comprising said anti-CD25 agent when the tumor immune phenotype in the sample from the patient is characterized as inflamed phenotype; and
d) recommending the start or continuation of therapy comprising said anti-CD25 agent.

In another embodiment, the above described method is an in vitro method.

In one embodiment, the recommendation in d) can be made at baseline, i.e. prior to starting the therapy and for selecting therapy with said anti-CD25 agent. In another embodiment the recommendation in d) can be made during treatment comprising said anti-CD25 agent in order to decide whether the treatment should be continued (treatment monitoring). In yet another embodiment, the therapy in step d) can also comprise one or several additional therapeutically active agents in combination with the anti-CD25 agent. In one embodiment said combination comprises at least one additional active ingredient, preferably a PD-1 or PD-L1 inhibitor authorized for use in humans. In one embodiment, said PD-L1 inhibitor is the antibody with the INN atezolizumab.

In another embodiment, the present invention provides a method of treating a patient suffering from cancer, said method comprises
e) detecting the tumor immune phenotype in a sample from the patient;
f) comparing the result measured in e) to a reference level;
g) identifying the patient as likely to respond to the therapy comprising an anti-CD25 agent when the tumor immune phenotype in the sample from the patient is characterized as inflamed phenotype; and
h) administering an anti-CD25 agent to said patient.

In one embodiment, the administration in step h) can be a monotherapy comprising an anti-CD25 agent, or can also comprise administration of one or several additional therapeutically active agents in combination with the anti-CD25 agent. In one embodiment said combination therapy comprises at least one additional active ingredient, preferably a PD-1 or PD-L1 inhibitor authorized for use in humans. In one embodiment, said PD-L1 inhibitor is the antibody with the INN atezolizumab. In another embodiment, the present invention provides a method of monitoring efficacy of a therapy comprising an anti-CD25 antibody in a patient having cancer, the method comprising:
i) detecting the tumor immune phenotype in a sample from the patient after the start of treatment comprising an anti-CD25 agent;
k) comparing the result measured in i) to a reference level, for example, the immune phenotype in a sample from that same patient at baseline;
l) adapting the treatment, such as e.g. terminate treatment or adapt the dose of said anti-CD25 agent when the comparison in k) reveals a difference to the value detected at baseline.

In one embodiment said comparison in k) may be a shift towards another immune phenotype, for example, a phenotype other than inflamed

In yet another embodiment, the present invention provides an anti-CD25 agent for use in treating a patient having cancer, wherein the patient is selected for treatment when the tumor immune phenotype as detected in a sample from the patient at baseline (i.e. prior to treatment with said anti-CD25 agent) is identified as inflamed.

In yet another embodiment, the present invention provides the in vitro identification of the tumor immune phenotype for assessing therapy comprising an anti-CD25 agent in a patient having cancer, wherein identification of an inflamed phenotype indicates that the patient should be treated with a therapeutically effective amount of an anti-CD25 agent. In one embodiment, said assessment is made at baseline, i.e. prior to starting the therapy with said anti-CD25 agent and will assist in the decision whether to start treatment with an anti-CD25 agent. In another embodiment said assessment is made during therapy with said anti-CD25 agent and will assist in the decision whether to continue treatment with an anti-CD25 agent.

In yet another embodiment, the present invention provides the in vitro use of identifying the tumor immune phenotype in sample from a patient with cancer for selecting that patient as likely to respond to a therapy comprising an anti-CD25 agent, wherein the identification of said phenotype as inflamed means that the patient is likely to respond to the therapy. In one embodiment, said identification of said inflamed phenotype is made at baseline, i.e. prior to starting the therapy with said anti-CD25 agent. In another embodiment, said identification of said inflamed phenotype is made during the therapy with said anti-CD25 agent.

In yet another embodiment, the present invention provides the use of the identification of the tumor immune phenotype as being inflamed, in a sample from a patient having cancer, for the manufacture of a diagnostic assay to assist in making the decision for treating said patient with a therapy comprising an anti-CD25 agent.

In one embodiment said sample is analyzed at baseline (i.e. prior to treatment with an anti-CD25 agent) and may assist in making the decision to start the treatment. In another embodiment said sample is analyzed during treatment with an anti-CD25 agent, and may assist in making the decision to continue the treatment. Any suitable assay platform known to the skilled person can be used. In one embodiment such assay is an in vitro assay.

In yet another embodiment, the present invention provides the use of the identification of the tumor immune phenotype as being inflamed for the manufacture of a diagnostics for assessing the likelihood of a response of a patient having cancer to a therapy comprising an anti-CD25 agent.

### Definitions

The term “cancer” as used herein means any type of hyper proliferative disease and is well known to a person of skill in the art, for example, an oncologist. In some embodiments a cancer in accordance with the present invention is a solid tumor. As used herein, the term “solid tumor” refers to an abnormal mass of tissue that usually does not contain cysts or liquid areas. Solid tumors may be benign or malignant. Different types of solid tumors are named for the type of cells that form them. Examples of solid tumors are sarcomas (including cancers arising from transformed cells of mesenchymal origin in tissues such as cancellous bone, cartilage, fat, muscle, vascular, hematopoietic, or fibrous connective tissues), carcinomas (including tumors arising from epithelial cells), melanomas, lymphomas, mesothelioma, neuroblastoma, retinoblastoma, etc. Cancers involving solid tumors include, without limitations, brain cancer, lung cancer, stomach cancer, duodenal cancer, esophagus cancer, breast cancer, colon and rectal cancer, renal cancer, bladder cancer, kidney cancer, pancreatic cancer, prostate cancer, ovarian cancer, melanoma, mouth cancer, sarcoma, eye cancer, thyroid cancer, urethral cancer, vaginal cancer, neck cancer, lymphoma, and the like. In one embodiment, the term cancer means breast cancer, colorectal cancer, head and neck squamous cell carcinoma (HNSCC), melanoma, non-small cell lung cancer (NSCLC), ovarian cancer and renal cancer. In another embodiment, the term cancer means head and neck squamous cell carcinoma (HNSCC), melanoma and non-small cell lung cancer (NSCLC).

As used herein, the term “therapeutically effective amount” means an amount (e.g., of an agent or of a pharmaceutical composition) that is sufficient, when administered to a population suffering from or susceptible to a disease and/or condition in accordance with a therapeutic dosing regimen, to treat such disease and/or condition. A therapeutically effective amount is one that reduces the incidence and/or severity of, stabilizes, and/or delays onset of, one or more symptoms of the disease, disorder, and/or condition. Those of ordinary skill in the art will appreciate that a “therapeutically effective amount” does not in fact require successful treatment be achieved in a particular subject.

As used herein, the term “treatment” (also “treat” or “treating”) refers to any administration of a substance (e.g. an anti-CD25 agent, as defined herein or as exemplified in WO2019/175222) that partially or completely alleviates, ameliorates, relives, inhibits, delays onset of, reduces severity of, and/or reduces incidence of one or more symptoms. In some embodiments, treatment may involve the direct administration of anti-CD25 agent, for example, as an injectable, aqueous composition, optionally comprising a pharmaceutically acceptable carrier, excipient and/or adjuvant, for use for intravenous, intratumoral or peritumoral injection. In some embodiments, the terms “respond to treatment” and “benefit from treatment” may have the same meaning.

A method described herein as “comprising” one or more named elements or steps is open-ended, meaning that the named elements or steps are essential, but other elements or steps may be added within the scope of the composition or method. It is also understood that any composition or method described as “comprising” (or which “comprises”) one or more named elements or steps, also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method.

The term “agent” as used herein, for example in connection with “anti-CD25 agent”, refers to a compound or entity of any chemical class including, for example, polypeptides, nucleic acids, small molecules or combinations thereof. Specific embodiments of agents that may be utilized in accordance with the present invention include small molecules, drugs, hormones, antibodies, antibody fragments, aptamers, nucleic acids (e.g., siRNAs, shRNAs, antisense oligonucleotides, ribozymes), peptides, peptide mimetics, etc. In one embodiment an “agent” is an antibody.

As used herein, the term “antibody” refers to a polypeptide that includes canonical immunoglobulin sequence elements sufficient to confer specific binding to a particular target antigen, such as CD25, human CD25 in particular, and human CD25 extracellular domain.

The anti-CD25 agents (or anti-CD25 antibodies) of the present invention are non-IL-2 blocking antibodies. The term “Non-IL-2 blocking antibody” is used herein to refer to those anti-CD25 antibodies (e.g. anti-CD25 non-IL-2 blocking antibodies) that are capable of specific binding to the CD25 subunit of the IL-2 receptor without blocking the binding of IL-2 to CD25 or signaling of IL-2 via CD25. In one embodiment, the anti-CD25 antibodies allow at least 50% of IL-2 signaling in response to IL-2 binding to CD25 compared to the level of signaling in the absence of the anti-CD25 antibody. Preferably, an anti-CD25 antibody in accordance with the present invention allows at least 75% of IL-2 signaling in response to CD25 compared to the level of signaling in the absence of the anti-CD25 Antibody.

In one embodiment an anti-CD25 agent (or anti-CD25 antibody) in accordance with the present invention is an antibody consisting of—or comprising specific sequences as disclosed in WO2018/167104 or WO2019/175222.

In another embodiment, anti-CD25 antibodies according to the present invention are antibodies having the sequence of “aCD25-a-686” in WO2019/175222, and more in general, antibodies that are or comprise one or more antigen-binding fragments or portions thereof, for example that comprise the aCD25-a-686-HCDR3 amino acid sequence as variable heavy chain complementarity determining region 3, and/or, in some embodiments, comprise one or both of the aCD25-a-686 HCDR1 and HCDR2 sequences as disclosed in WO2019/175222. Anti-CD25 antibodies in accordance with the present invention include the affinity matured variants aCD25-a-686-m1, aCD25-a-686-m2, aCD25-a-686-m3, aCD25-a-686-m4 and aCD25-a-686-m5, as also disclosed in WO2019/175222.

In another embodiment, an anti-CD25 antibody in accordance with the present invention is an antibody that comprises the HCDR1, HCDR2 and HCDR3 amino acid sequences of aCD25-a-686 as disclosed in WO2019/175222. In another embodiment, an anti-CD25 antibody in accordance with the present invention is an antibody that comprises the HCDR1, HCDR2, HCDR3, LCDR1, LCDR2 and LCDR3 amino acid sequences of aCD25-a-686 as disclosed in WO2019/175222. In another embodiment, an anti-CD25 antibody in accordance with the present invention is an antibody that comprises the variable heavy amino acid sequence of aCD25-a-686 as disclosed in WO2019/175222. In another embodiment, an anti-CD25 antibody in accordance with the present invention is an antibody that comprises the variable heavy and variable light amino acid sequences of aCD25-a-686 as disclosed in WO2019/175222, or of a variant thereof.

In another embodiment, an anti-CD25 Antibody in accordance with the present invention means an antibody that competes with aCD25-a-686 (or an antigen-binding fragment or derivative or variant thereof, including affinity matured variants as disclosed in WO2019/175222) for binding to human CD25 extracellular domain.

In another embodiment, an anti-CD25 agent in accordance with the present invention is the antibody used in the Phase 1 clinical trial with ClinicalTrials.gov Identifier: NCT04158583, and which is also designated RG6292.

In still another embodiment, the anti-CD25 agent is an IgG1 antibody, preferably a human IgG1 antibody, which is capable of binding to at least one Fc activating receptor. For example, the antibody may bind to one or more receptor selected from FcγRI, FcγRIIa, FcγRIIc, FcγRIIIa and FcγRIIIb. In some embodiments, the antibody is capable of binding to FcγRIIIa. In some embodiments, the antibody is capable of binding to FcγRIIIa and FcγRIIa and optionally FcγRI. In some embodiments, the antibody is capable of binding to these receptors with high affinity, for example with a dissociation constant of less than about 10-7M, 10-8M, 10-9M or 10-10M. In some embodiments, the antibody binds an inhibitory receptor, FcγRIIb, with low affinity. In one aspect, the antibody binds FcγRIIb with a dissociation constant higher than 10-7M, higher than 10-6M or higher than 10-5M.

In yet another embodiment in accordance with the present invention, an anti-CD25 agent is an antibody comprising the heavy chain variable domain (VH) and light chain variable domain (VL) as shown by the sequences No's 1 and 2 in Table 1.

TABLE 1

| Sequence | |
|---|---|
| ID No. | Sequence (protein) |
| 1 (VH) | QVQLVQSGAEVKKPGSSVKVSCKASGGTFSSLAISWVRQA |
| | PGQGLEWMGGIIPIFGTANYAQKFQGRVTITADESTSTAY |
| | MELSSLRSEDTAVYYCARGGSVSGTLVDFDIWGQGTMVTV |
| | SS |
| 2 (VL) | DIQMTQSPSTLSASVGDRVTITCRASQSISSWLAWYQQKP |
| | GKAPKLLIYKASSLESGVPSRFSGSGSGTEFTLTISSLQP |
| | DDFATYYCQQYNIYPITFGGGTKVEIK |

In yet another embodiment in accordance with the present invention, an anti-CD25 agent is an antibody that competes for binding to human CD25 with the antibody comprising the VH and VL of Table 1 herein.

In yet another embodiment in accordance with the present invention, an anti-CD25 agent is an antibody comprising the heavy chain (HC)—and light chain (LC) amino acid sequences as shown by the sequences No's 3 and 4 in Table 2.

TABLE 2

| Sequence | |
|---|---|
| ID No. | Sequence (protein) |
| 3 (HC) | QVQLVQSGAE VKKPGSSVKV SCKASGGTFS SLAISWVRQA |
| | PGQGLEWMGG IIPIFGTANY AQKFQGRVTI TADESTSTAY |
| | MELSSLRSED TAVYYCARGG SVSGTLVDFD IWGQGTMVTV |
| | SSASTKGPSV FPLAPSSKST SGGTAALGCL VKDYFPEPVT |
| | VSWNSGALTS GVHTFPAVLQ SSGLYSLSSV VTVPSSSLGT |
| | QTYICNVNHK PSNTKVDKKV EPKSCDKTHT CPPCPAPELL |
| | GGPSVFLFPP KPKDTLMISR TPEVTCVVVD VSHEDPEVKF |
| | NWYVDGVEVH NAKTKPREEQ YNSTYRVVSV LTVLHQDWLN |
| | GKEYKCKVSN KALPAPIEKT ISKAKGQPRE PQVYTLPPSR |
| | DELTKNQVSL TCLVKGFYPS DIAVEWESNG QPENNYKTTP |
| | PVLDSDGSFF LYSKLTVDKS RWQQGNVFSC SVMHEALHNH |
| | YTQKSLSLSP GK |
| 4 (LC) | DIQMTQSPST LSASVGDRVT ITCRASQSIS SWLAWYQQKP |
| | GKAPKLLIYK ASSLESGVPS RFSGSGSGTE FTLTISSLQP |
| | DDFATYYCQQ YNIYPITFGG GTKVEIKRTV AAPSVFIFPP |
| | SDEQLKSGTA SVVCLLNNFY PREAKVQWKV DNALQSGNSQ |
| | ESVTEQDSKD STYSLSSTLT LSKADYEKHK VYACEVTHQG |
| | LSSPVTKSFN RGEC |

The term “sample” as used herein means a patient's tumor biopsy sample.

The term “inflamed” in connection with a “tumor” or “tumor phenotype” or “immune phenotype” as used herein is well known to the skilled person, and is for example defined in (9, 10) together with “immune-desert” or “immune-excluded” (tumor) phenotypes.
Immune-desert tumors: These tumors have no or very few T cells. Usually there is a total lack of an immune response in the tumour.
Immune-excluded tumors: T cells are present close to the tumor but they cannot penetrate into the tumor microenvironment.
Inflamed tumors: Activated T cells are present within the tumor and are capable of recognizing upon appropriate stimuli the tumor and attack it. Although inhibitory factors might still be capable of preventing this active immune response from actually completely destroying all of the cancer cells.

The phenotypes classification is based on the location of the immune cells in the tumor cells nest (inflamed phenotype) or the stroma (excluded) phenotype. Tumors that have a low number of immune cells are classified as immune deserts. In a bladder cancer study mUC, a large proportion of tumors (47%) exhibited the excluded phenotype; by contrast, only 26% and 27% of tumors exhibited the inflamed and desert phenotypes, respectively (9). The mean signal for the CD8+Teff signature was lowest in the desert phenotype, intermediate in the excluded phenotype and highest in inflamed tumors, and was significantly associated with response only in inflamed tumors. It was estimated that the interferon-gamma signature best correlated with a tumor area that showed an infiltration of the 20% or more of the tumor strands in the tumor area (9, 10).

In accordance with the present invention, patients having cancer are likely to benefit from treatment with an anti-CD25 agent if they show an inflamed tumor phenotype. The term “reference” (or “reference level”), as used herein, for making that comparison may be any of the other established phenotypes (i.e. immune-desert and immune-excluded). In one embodiment, the term inflamed means that activated T cells, preferably activated CD8 cells, are present within the tumor (intratumoral) and are capable of recognizing, upon appropriate stimuli, the tumor and attack it. CD8 T cells can be detected in the tumor by several known methodologies such as by Immunohistochemistry, Immunofluorescence, multiplex approaches and others (11, 12, 13, 14).

### EXAMPLES

Materials and Methods

All samples used in the analyses described herein, are from patient's tumor biopsy samples used for participation of the patients to clinical trials undertaken by F. Hoffmann-La Roche AG (herein Roche, with offices in Basel, Switzerland) and/or its affiliated companies. All samples were used prior to any new investigational drug being administered.

Distribution of CD8+ immune phenotypes were assessed using established methods to distinguish inflamed, desert and excluded tumor phenotypes (9, 10).
Immune deserts: These tumors have no or very few T cells. Usually there is a total lack of an immune response in the tumour.
Immune-excluded tumours: T cells are present close to the tumor but they cannot penetrate into the tumour microenvironment.
Inflamed tumours: Activated T cells are present within the tumor and are capable of recognizing upon appropriate stimuli the tumor and attack it. Although inhibitory factors might still be capable of preventing this active immune response from actually completely destroying all of the cancer cells.

The phenotypes classification is based on the location of the immune cells in the tumor cells nest (inflamed phenotype) or the stroma (excluded) phenotype. Tumors that have a low number of immune cells are classified as immune deserts. In a bladder cancer study mUC, a large proportion of tumours (47%) exhibited the excluded phenotype; by contrast, only 26% and 27% of tumours exhibited the inflamed and desert phenotypes, respectively (9). The mean signal for the CD8+ Teff signature was lowest in the desert phenotype, intermediate in the excluded phenotype and highest in inflamed tumours, and was significantly associated with response only in inflamed tumours. It was estimated that the interferon-gamma signature best correlated with a tumor area that showed an infiltration of the 20% or more of the tumor strands in the tumor area (9, 10).

CD8 T cells can be detected in the tumor by several methodologies such as by Immunohistochemistry, Immunofluorescence, multiplex approaches and others (11, 12, 13, 14).

### Example 1

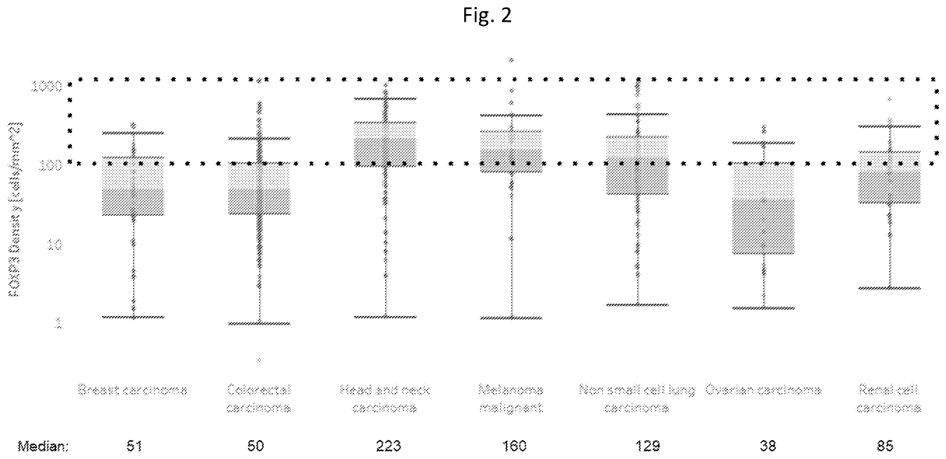

Applicant's (Roche) internal tissue/sample databases were investigated with respect to FOXP3 content by IHC, immune phenotype by IHC and mRNA expression data.
a) Samples with available FOXP3 staining were used to assess FOXP3 level variation across indications. The inventors analysed BC (n=72), CRC (n=344), Melanoma (n=46), NSCLC (n=101), OvC (n=59) and RCC (n=37) samples that were stained with FOXP3 (FIG. 2)
b) Samples with available immune phenotype classification were included in the analyses described below regarding strongest predictive surrogate biomarker.
c) Samples with available RNASeq gene expression profile were included in the analyses described below regarding strongest predictive surrogate biomarker

### Example 2

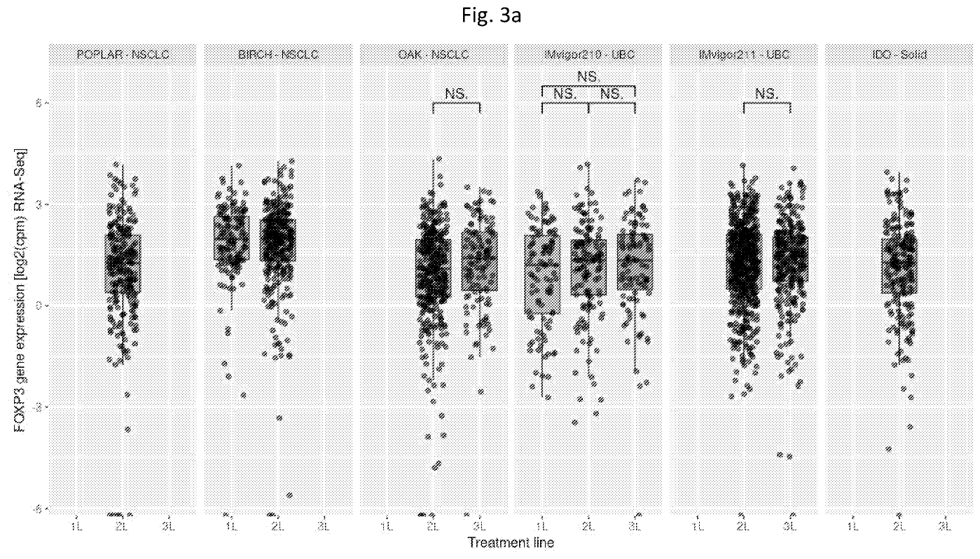

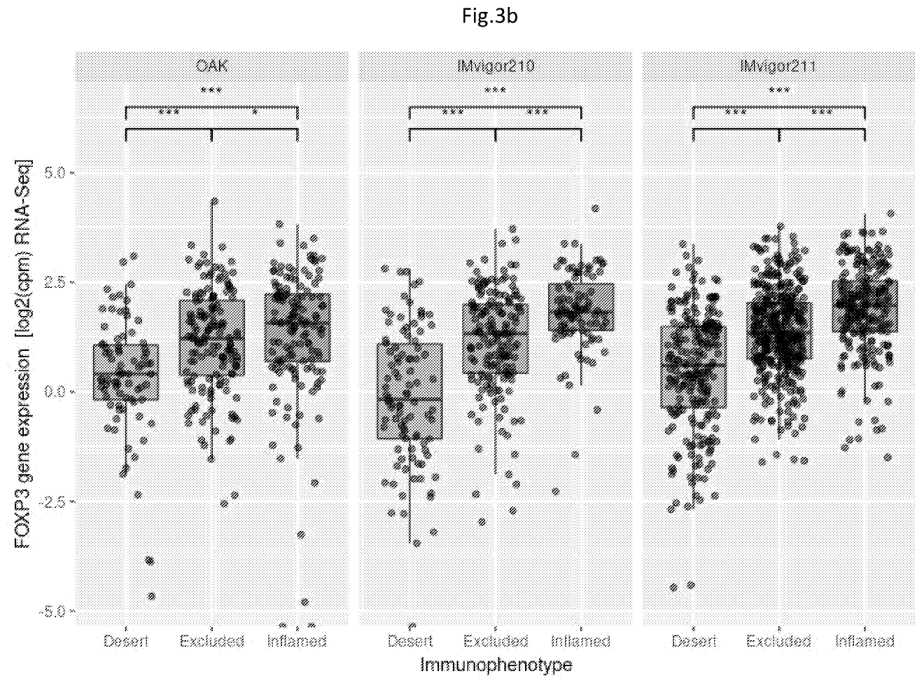

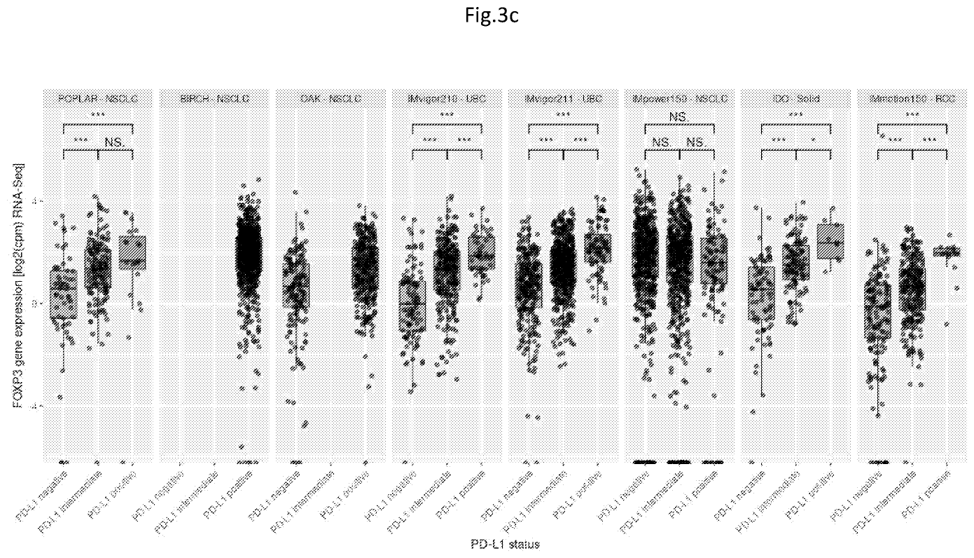

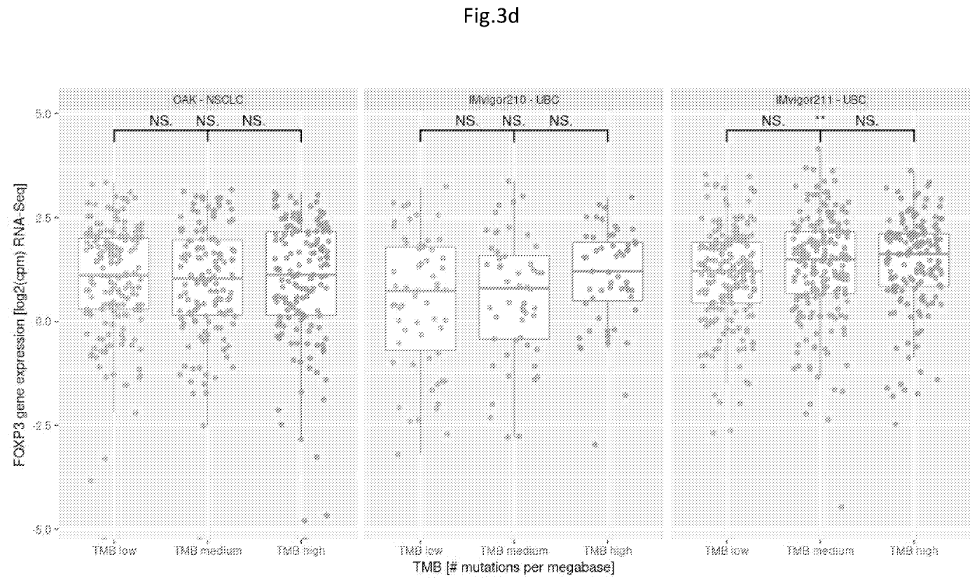

Samples from Roche internal tissue/sample databases were interrogated with respect to relation of FOXP3 and CD8 expression against line of treatment, PDL1 expression status, Immune phenotype and TMB status as described below (FIG. 3).

The inventors obtained RNASeq gene expression data from all patients from 3 Roche trials (namely OAK, IMvigor210 and IMvigor211). The expression values were normalized to counts-per-million (cpm) and log transformed. In addition, clinical variables from the internal EDIS CIT Datamart database were obtained, such as the indication, line of treatment (LoT), PD-L1 status, tumor mutational burden (TMB), as well as the immunophenotypes (inflamed, excluded, deserted) as annotated by Histogenex (15) for all patient samples. T regulatory cell (Treg) expression was evaluated using CD25 and FOXP3 RNASeq gene expression as proxies.

Results

FOXP3 cell numbers (median) across indications showed high variability with the highest observed in HNSCC, Melanoma and NSCLC

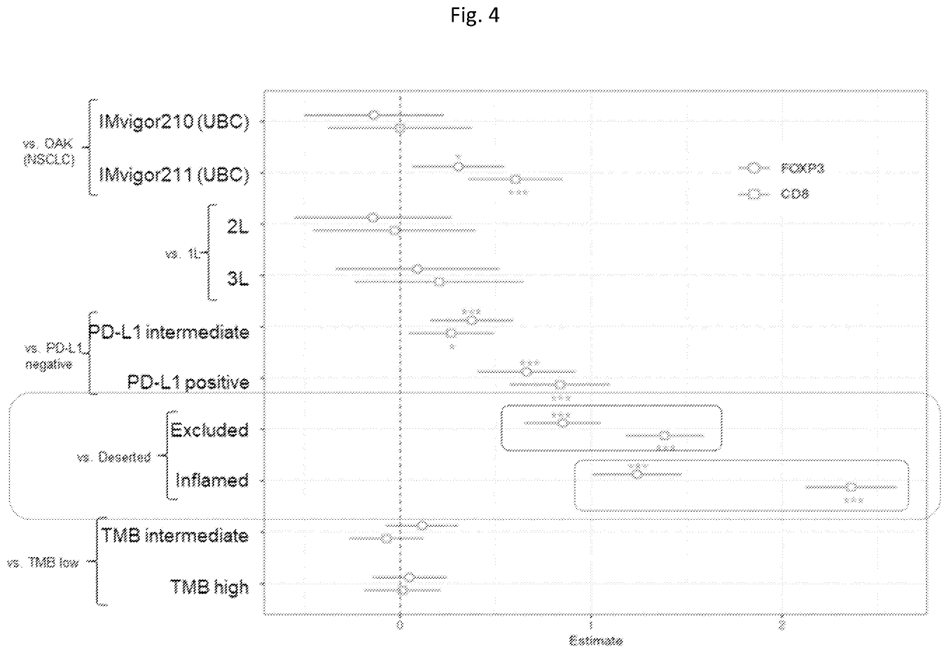

The inventors investigated differences in Treg expression levels univariately for all clinical variables by plotting the distribution of expression values for each factor within a clinical variable (e.g. inflamed vs. excluded vs. deserted) and quantified the differences using Wilcoxon's rank sum test. Additionally, the inventors modeled Treg expression (i.e. CD25 and FOXP3 gene expression) using a linear model across all available clinical variables. Compared against the reference (study=OAK, LoT=1 L, PD-L1=negative, immunophenotype=desert, TMB=low), a significant positive association between high Treg expression (FOXP3 gene expression) and the following factors was identified: study=IMvigor210, study=IMvigor211, PD-L1=intermediate, PD-L1=high, immunophenotype=excluded, immunophenotype=inflamed. The strongest association was observed between high Treg expression and inflamed immunophenotypes (FIG. 4).

### REFERENCES

(1) Galon J, Costes A, Sanchez-Cabo F, Kirilovsky A, Mlecnik B, Lagorce-Pages C, et al. Type, density, and location of immune cells within human colorectal tumors predict clinical outcome. Science 2006; 313(5795):1960-4.
(2) Hegde P S, Chen D S. Top 10 Challenges in Cancer Immunotherapy. Immunity. 2020; 52:17-35.
(3) Gajewski T F. The next hurdle in cancer immunotherapy: overcoming the non-T-cell-inflamed tumor microenvironment. Semin Oncol 2015; 42:663-71.
(4) Sharma P, Allison J P. The future of immune checkpoint therapy. Science 2015; 348:56-61.
(5) Kandoth C et al Mutational landscape and significance across 12 major cancer types. Nature 2013; 502:333-9.
(6) Hegde P S, Karanikas V, Evers S. The Where, the When, and the How of Immune Monitoring for Cancer Immunotherapies in the Era of Checkpoint Inhibition. Clin Cancer Res 2016; 22:1865-74.
(7) Chen, D and Mellman I. Oncology Meets Immunology: The Cancer-Immunity Cycle. Immunity 2013; 39(1): 1-10.
(8) Powles T, Kockx M, Rodriguez-Vida A, Duran I, Crabb S J, Van Der Heijden M S, et al. Clinical efficacy and biomarker analysis of neoadjuvant atezolizumab in operable urothelial carcinoma in the ABACUS trial. Nat Med. 2019; 25:1706-1714.
(9) Mariathasan, S. et al. TGF-β attenuates tumour response to PD-L1 blockade by contributing to exclusion of T cells. Nature 554, 544-548 (2018).
(10) Powles, T. et al. Atezolizumab (atezo) vs. chemotherapy (chemo) in platinum-treated locally advanced or metastatic urothelial carcinoma (mUC): immune biomarkers, tumor mutational burden (TMB), and clinical outcomes from the phase III IMvigor211 study. J. Clin. Oncol. 2018:36, 409-409.
(11) Galon J, Bruni D. Approaches to treat immune hot, altered and cold tumours with combination immunotherapies. Nat Rev Drug Discov 2019; 18:197-218.
(12) Zhang W, Hubbard A, Jones T, Racolta A, Bhaumik S, Cummins N, et al. Fully automated 5-plex fluorescent immunohistochemistry with tyramide signal amplification and same species antibodies. Lab Invest 2017; 97:873-85.
(13) Decalf J, Albert M L, Ziai J. New tools for pathology: a user's review of a highly multiplexed method for in situ analysis of protein and RNA expression in tissue. J Pathol 2019; 247:650-61.
(14) Navas T, Fino K, Fung K L, Cutuli F, Kinders R J, Sharma A et al. A multiplex immunofluorescence assay to assess immune checkpoint inhibitor-targeted CD8 activation and tumor colocalization in FFPE tissues. J Clin Oncol 2019; 37:15_suppl, 2629.
(15) www.histogenex.com/immunotherapy-biomarkers

1. A biomarker for identifying patients likely to benefit from treatment with an anti-CD25 agent, wherein such biomarker consists in identifying the immune phenotype of a patient's tumor as being the inflamed immune phenotype

2. A method of identifying a patient having cancer as likely to respond to a therapy comprising an anti-CD25 agent, the method comprising:
a) detecting the tumor immune phenotype in a sample from the patient;
b) comparing the result measured in a) to a reference level;
c) identifying the patient as likely to respond to the therapy comprising said anti-CD25 agent when the tumor immune phenotype in the sample from the patient is characterized as inflamed phenotype; and
d) recommending the start or continuation of therapy comprising said anti-CD25 agent.

3. A method of treating a patient suffering from cancer, the method comprising:
a) detecting the tumor immune phenotype in a sample from the patient;
b) comparing the result measured in a) to a reference level;
c) identifying the patient as likely to respond to a therapy comprising an anti-CD25 agent when the tumor immune phenotype in the sample from the patient is characterized as inflamed phenotype; and
d) administering the anti-CD25 agent to said patient.

4. An anti-CD25 agent for use in treating a patient having cancer, wherein the patient is selected for treatment when the tumor immune phenotype as detected in a sample from the patient at baseline is identified as inflamed.

5. The anti-CD25 agent for use according to claim 4, wherein the anti-CD25 agent is a non-IL2 blocking anti-CD25 antibody.

6. The anti-CD25 antibody for use according to claim 5, wherein the anti-CD25 antibody is a human or humanized IgG1 antibody.

7. The anti-CD25 antibody for use according to claim 5, wherein the anti-CD25 antibody comprises the heavy chain variable domain (VH) of SEQ ID NO:1 and the light chain variable domain (VL) of SEQ ID NO:2.

8. The anti-CD25 antibody for use according to claim 6, wherein the anti-CD25 antibody comprises the heavy chain variable domain (VH) of SEQ ID NO:1 and the light chain variable domain (VL) of SEQ ID NO:2.

9. The method for identifying a patient having cancer according to claim 2, wherein the cancer is a solid tumor.
